(19) United States
(12) Patent Application Publication
(10) Pub. No.: US 2010/0204525 A1
(43) Pub. Date: Aug. 12, 2010

(54) CATALYSTS FOR HYDROGEN PRODUCTION FOR LOW TEMPERATURE FUEL CELLS BY STEAM REFORMING AND AUTOTHERMAL REFORMING OF ALCOHOLS

(75) Inventor: Fabio Bellot NORONHA, Niteroi (BR)

Correspondence Address: ANDERSON TMP , INC, 1903 60 TH PLACE E, SUITE M5103, BRADENTON, FL, 34203, US

(73) Assignee: INSTITUTO NACIONAL DE TECHNOLOGIA-INT, Rio de Janeiro- Saude (BR)

(21) Appl. No.: 12/669,569

(22) PCT Filed: Dec. 14, 2007
(86) PCT No.: PCT/BR2007/000337

§ 371 Date: Jan. 19, 2010

(30) Foreign Application Priority Data
Jul. 19, 2007 (BR) PI 0703296-0

(51) Int. Cl. C07C 31/08; C01F 17/00; B01J 23/10

(52) U.S. Cl. 568/840; 423/263; 502/304; 502/303

(57) ABSTRACT

The present invention involves the use of the cerium oxide based catalysts with or without 0.5-10 wt % of alkaline and alkaline earth promoters (Li, Na, K, Rb, Cs, Fr, Be, Mg, Ca, Sr, Ba, Ra) and mixed oxides containing ceria and zirconia and/or yttria an/or lanthanide elements (CeₓM₁₋ₓO₂; M=Zr, Y, La, Pr, Nd, Pm, Sm, Eu and 0.1<x<0.9) on the steam reforming and autothermal reforming at low temperatures of alcohols, in particular ethanol, or a mixture of these alcohols, like, for example, bio-ethanol. Low temperature was defined as 723-823 K. The catalysts of this invention exhibit good activity and stability, high selectivity to hydrogen, low formation of carbon monoxide (<150 ppm), small amounts of acetaldehyde and ethene and no production of ketone.

### FIELD OF THE INVENTION

This invention comprises the use of the cerium oxide based catalysts with or without alkaline and alkaline earth promoters and mixed oxides containing ceria and zirconia and/or elements of lanthanide group in the steam reforming and autothermal reforming at low temperatures of alcohols, in particular ethanol, or a mixture of these alcohols, like, for example, bio-ethanol. These catalysts presented high activity, high stability and high selectivity to hydrogen (without significant formation of CO) in the reactions described above.

Nowadays, hydrogen has been proposed as a major energy source that could contribute to the reduction of global dependence on fossil fuels, greenhouse gas emissions and atmospheric pollution.

Hydrogen-powered fuel cells represent a radically different approach to energy conversion. These systems directly convert chemical energy into electric power, without the intermediate production of mechanical work, and they are more efficient than the conventional combustion engines [Amphlett et al, Int J. Hydrogen Energy 19 (1994) 131; Hirschenhofer et ah, Fuel Cell Handbook, 1998]. There are several types of fuel cells which differ in the type of electrolyte and in the temperature of operation. Proton exchange membranes fuel cells (PEMFC) operate at low temperatures (˜373 K) and offer large power density along with fast response times [Hirschenhofer et ah, Fuel Cell Handbook, 1998].

Hydrogen for fuel cells can be derived from a variety of energy sources such as gasoline, diesel, LPG, methane, and alcohols, in particular ethanol. For example, the bio-ethanol obtained through biomass has been proposed as a promising renewable source of hydrogen for these systems that address the issue of the greenhouse effect. Furthermore, in countries like Brazil, the use of bio-ethanol has an additional advantage since the infrastructure needed for ethanol production and distribution is already established. However, the hydrogen production from ethanol present some disadvantages such as the formation of by-products and the deactivation of catalysts [Guil et al., Phys. Chem. B 109 (2005) 10813; Takezawa & Iwasa, Catal. Today 36 (1997) 45; Cavallaro, Mondello & Freni, J. Power Sources 102 (2001) 198]. Another problem related to the use of bio-ethanol is the high costs of ethanol concentration process from the aqueous solution derived from fermentation, which contains approximately 10 wt % of ethanol per volume of solution (H₂O/ethanol molar ratio=23) [Vargas et al, Catal Today 107 (2005) 417]. Then, the development of catalysts that exhibit good performance under a feedstock containing high H₂O/ethanol molar ratio could reduce the costs of the use of bio-ethanol as a source of hydrogen for fuel cells, since the distillation process could be eliminated.

Hydrogen for fuel cells can be produced by steam reforming of alcohols [J. C. Vargas, S. Libs, A. Roger, A. Kiennemann, Catal Today 107 (2005) 417, N. Takezawa, N. Iwasa, Catal. Today 36 (1997) 45; S. Cavallaro, N. Mondello, S. Freni, J. Power Sources 102 (2001) 198; J. C. Vargas, S. Libs, A. Roger, A. Kiennemann, Catal Today 107 (2005) 417; N. Takezawa, N. Iwasa, Catal. Today 36 (1997) 45; S. Cavallaro, N. Mondello, S. Freni, J. Power Sources 102 (2001) 198; F J. Marino, E. G. Cerrela, S. Duhalde, M. Jobbagy, M. A. Laborde, Int. J. Hydrogen Energy 12 (1998) 1095; F J. Marino, M. Boveri, G. Baronetti, M. Laborde, Int. J. Hydrogen Energy 26 (2001). 665; E. Y. Garcia, M. A Laborde, Int. J. Hydrogen Energy 16 (1991). 3O7; S. Freni, N. Mondello, S. Cavallaro, G. Cacciola, V. N. Parmon, V. A. Sobyanin, React. Kinet. Catal. Lett. 71, (2000)143; V V. Galvita, G. L. Semin, V. D. Belyaev, V. A. Semikolenov, P. Tsiakaras, Sobyanin, Appl. Catal. A: General 220 (2001). 123; A. N. Fatsikostas, D. I. Kondarides, X. E. Verykios, Chem. Commun. 851 (2001); A. N. Fatsikostas, D. I. Kondarides, X. E. Verykios, Catal. Today 75 (2002) 145; J. P. Breen, R. Burch, H. M. Coleman, Appl. Catal. B. 39 (2002) 65; J. Llorca, N. Horns, J. Sales, P. R. de Ia Piscina, J. Catal 209 (2002) 306; J. Comas, F. Marino, M. Laborde, N. Amadeo, Chem. Eng. J., 98 (2004) 61; H. V. Fajardo, L. F. D. Probst, Appl. Catal. A 306 (2006) 134; E. G. Wanat, K. Venkataraman, L. D. Schmidt, Appl. Catal. A 276 (2004) 155; F. Frusteri, S. Freni, V. Chiodo, L. Spadaro, O. Di Blasi, G. Bonura, S. Cavallaro, Appl. Catal. A 270 (2004) 1] and autothermal reforming of alcohols [Vessellia et al, Appl. Catal. A 281 (2005) 13922-26; Navarro et al, Appl. Catal. B, 55 (2005) 229; Velu et al, Catal. Letters 82 (2002) 145; Kugai, Velu & Song, Catal Letters 101 2005 255; Deluga et al, Science 303 (2004) 13].

Steam reforming of alcohols like, for example, ethanol (equation 1) is an endothermic reaction. Then, the addition of energy to the system is necessary, which leads to high capital and operation costs. [A. Haryanto, S. Fernando, N. Murali, S. Adhikari, Energy & Fuels 19 (2005) 2098]. One alternative way of supplying heat to the system is to add oxygen or air to the feedstock and simultaneously to burn a portion of ethanol, reaching the thermal neutrality of the reaction [D. K. Liguras, K. Goundani, X. E. Verykios, Int. J. Hydrogen Energy 29 (2004) 419]. This process is called autothermal reforming. The autothermal reforming of ethanol is described by the equation 2 [D. K. Liguras, K. Goundani, X. E. Verykios, Int. J. Hydrogen Energy 29 (2004) 419].

C₂H₅OH+3H₂O→2CO₂+6H₂(ΔH°298 =+347.4 kJ/mol) (1)

C₂H₅OH+0.61O₂+1.78H₂O→2CO₂+4.78H₂(ΔH°298=0 kJ/mol) (2)

Nevertheless, several parallels reactions can occur on these two routes, depending on the catalysts and the reaction conditions used, hi particular, for ethanol, the following reactions can be observed:
(i) Dehydration of ethanol to ethene (equation 3), followed by polymerization of ethene to form coke (equation 4) [A. Haryanto, S. Fernando, N. Murali, S. Adhikari, Energy & Fuels 19 (2005) 2098].

C₂H₅OH→C₂H₄+H₂O (3)

C₂H₄→coque (4)
(ii) Decomposition of ethanol (equation 5), producing methane, carbon monoxide and hydrogen. The methane can react with water (steam reforming of methane), forming carbon monoxide and hydrogen (equation 6) [A. Haryanto, S. Fernando, N. Murali, S. Adhikari, Energy & Fuels 19 (2005) 2098].

C₂H₅OH→CH₄+CO+H₂ (5)

CH₄+H₂O→CO+3H₂ (6)
(iii) Dehydrogenation of ethanol, producing acetaldehyde (equation 7) [A. Haryanto, S. Fernando, N. Murali, S. Adhikari, Energy & Fuels 19 (2005) 2098].

C₂H₅OH→C₂H₄O+H₂ (7)
(iv) Formation of ketone (equation 8) [T. Nishiguehi, T. Matsumoto, H. Kanai, K. Utani, Y. Matsumurab, W-J. Shenc, S. Imamura, Appl. Catal. A 279 (2005) 273]

2C₂H₅OH+H₂O→CH₃COCH₃+CO₂+4H₂ (8)

The appropriated catalyst for reforming of alcohols should maximize the hydrogen production and minimize by-products formation. The majority of patents [US 2005/0244329; FR 2 857 003-A1; US 2003/0022950 A1; US 2001/0023034 A1; U.S. Pat. No. 6,387,554 B1; FR 2 795 339-A1; WO 99/61368; US 2005/0260123 A1; EP 1 314 688 B1; BE 898.686; US 2004/0137288 A1] and papers [N. Takezawa, Niwasa, Catal. Today 36 (1997) 45; S. Cavallaro, N. Mondello, S. Freni, J. Power Sources 102 (2001) 198; J. C. Vargas, S. Libs, A. Roger, A. Kiennemann, Catal Today 107 (2005) 417; N. Takezawa, N. Iwasa, Catal. Today 36 (1997) 45; S. Cavallaro, N. Mondello, S. Freni, J. Power Sources 102 (2001) 198; FJ. Marino. E. G. Cerrela, S. Duhalde, M. Jobbagy, M. A. Laborde, Int. J. Hydrogen Energy 12 (1998) 1095; F. J. Marino, M. Boveri, G. Baronetti, M. Laborde, Int. J. Hydrogen Energy 26 (2001). 665; E. Y. Garcia, M. A Laborde, Int. J. Hydrogen Energy 16 (1991). 307; S. Freni, N. Mondello, S. Cavallaro, G. Cacciola, V. N. Parmon, V. A. Sobyanin, React. Kinet. Catal. Lett. 71, (2000)143; V. V. Galvita, G. L. Semin, V. D. Belyaev, V. A. Semikolenov, P. Tsiakaras, Sobyanin, Appl. Catal. A: General 220 (2001). 123; A. N. Fatsikostas, D. I. Kondarides, X. E. Verykios, Chem. Commun. 851 (2001); A. N. Fatsikostas, D. I. Kondarides, X. E. Verykios, Catal. Today 75 (2002) 145; J. P. Breen, R. Burch, H. M. Coleman, Appl Catal. B. 39 (2002) 65; J. Llorca, N. Horns, J. Sales, P. R. de Ia Piscina, J. Catal 209 (2002) 306; J. Comas, F. Marino, M. Laborde, N. Atnadeo, Chem. Eng. J., 98 (2004) 61; H. V. Fajardo, L. F. D. Probst, Appl. Catal. A 306 (2006) 134; E. C. Wanat, K. Venkataraman, L. D. Schmidt, Appl. Catal. A 276 (2004) 155; F. Frusteri, S. Freni, V. Chiodo, L. Spadaro, O. Di Blasi, G. Bonura, S. Cavallaro, Appl. Catal. A 270 (2004)1; E. Vessellia, b, G. Comellia, R. Roseia, S. Frenic, F. Frusteric, S. Cavallaro, Appl. Catal. A 281 (2005) 139; R. M. Navarro, M. C. Alvarez-Galvana, M. C. Sanchez-Sancheza, F. Rosab, J. L. G. Fierro, Appl. Catal. B, 55 (2005) 229; S. Velu, N. Satoh, C. S. Gopinath, K. Suzuki, Catal. Letters 82 (2002) 145; J. Kugai, S. Velu, C. Song, Catal Letters 101 2005 255, G. A. Deluga, J. R. Salge, L. D. Schmidt, X. E. Verykios, Science 303 (2004) 13] found in the literature reported the use of supported metals as catalysts for steam reforming and autothermal reforming of alcohols. The majority of these catalysts showed better performance at high temperatures (between 873 and 1023 K). At low temperatures, the production of oxygenated products increases and the formation of coke is thermodynamically favored. On the other hand, at high temperatures, the thermodynamic equilibrium leads to the production of large amounts of CO (higher than 10 ppm), which poison the electrodes of PEM fuel cells. In order to ensure long and efficient use of hydrogen-fueled PEM fuel cell, highly pure hydrogen must be delivered. Then, water gas shift reaction and preferential oxidation of CO reaction or pressure swing adsorption steps are required for CO removal, as showed in FIG. 1.

During the WGS reaction, carbon monoxide is converted to carbon dioxide and hydrogen through a reaction with steam. Although the equilibrium of this reaction favors the products formation at lower temperatures, reaction kinetics are faster at higher temperatures [A. Haryanto, S. Fernando, N. Murali, S. Adhikari, Energy & Fuels 19 (2005) 2098]. Then, the water gas shift reaction is carried out in two steps (FIG. 1). At first, the reaction is performed at 623-643 K (high temperature shift —HTS). After this step, the reaction is carried out at 473-493 K (low temperature shift—LTS). At the end of the WGS reaction, the CO concentration is between 1.0 and 2.0 mol %. The WGS reaction is followed by preferential oxidation of CO reaction or pressure swing adsorption. The concentration of CO at the exit of this last step is around 10 ppm, which is appropriated to the PEM fuel cells.

Then, the development of the catalysts that exhibit high performance on the reforming of alcohols at low temperatures, producing low amounts of CO and by-products, could reduce the costs associated to the hydrogen purification steps, as described above.

Some works in the literature [J. M. Guil, N, Horns, J. Llorca, P. R. de Ia Piscina, J. Phys. Chem. B 109 (2005) 10813; A. Haryanto, S. Fernando, N. Murali, S. Adhikari, Energy & Fuels 19 (2005) 2098; A. N. Fatsikostas, X. E. Verykios, J. Catal 225 (2004) 439; T. Nishiguchi, T. Matsumoto, H. Kanai, K. Utani, Y. Matsumurab, W-J. Shenc, S. Imamura, Appl. Catal. A 279 (2005) 273; N. Laosiripojana, S. Assabumrungrat, Appl. Catal. B 66 (2006) 29; J. Llorca, N. Horns, P. R. de Ia Piscina, J. Catal 227 (2004) 556] showed that the use of oxides as catalysts for steam reforming and autothermal reforming of alcohols decreases the CO formation, which is not detected, depending on the reaction conditions used. However, when the activity of oxides and supported metal catalysts is compared, it was observed that the former presented lower ethanol conversion than the later. Furthermore, there was a significant formation of by-products such as ethene, acetaldehyde and ketone, over oxides based catalysts.

Al₂O₃ and La₂O₃ oxides exhibited low formation of hydrogen and production of large amounts of ethene and acetaldehyde on steam reforming and autothermal reforming of ethanol [A. N. Fatsikostas, X. E. Verykios, J. Catal 225 (2004) 439]. Moreover, it was detected carbon deposition on both oxides, mainly on alumina. The dehydration of ethanol and the dehydrogenation of ethanol reactions were favored over Al₂O₃ and La₂O₃, respectively.

The performance of CeO₂ on steam reforming of ethanol was evaluated only at 593 K [T. Nishiguchi, T. Matsumoto, H. Kanai, K. Utani, Y. Matsumurab, W-J. Shenc, S. Imamura, Appl. Catal. A 279 (2005) 273] and at 1173 K [N. Laosiripojana, S. Assabumrungrat, Appl. Catal. B 66 (2006) 29]. It was used a H2O/etanol molar ratio of 5 (593 K) and between 3 and 5 (1173 K). In spite of the large amount of catalyst used (450 mg), a low ethanol conversion (˜16%) was obtained at 593K. The main products observed were ketone and hydrogen. Moreover, small amounts of ethene were detected and it was not observed the acetaldehyde formation. No comments were done about the stability of this material and the production of carbon monoxide during the reaction. At high temperatures (1173 K), CeO₂ oxides with different BET surface areas (22.5 and 7.3 m²/g) were studied [N. Laosiripojana, S. Assabumrungrat, Appl. Catal. B 66 (2006) 29]. The reaction was carried out at high temperatures since the aim of the work was to produce hydrogen for solid oxides fuel cells (SOFCs), which only operate at elevated temperatures. The results showed that all samples exhibited complete ethanol conversion. Moreover, all catalysts were stable, when a H₂O/ethanol molar ratio of 3 was used. Concerning the products selectivity, CeO₂ oxide with BET surface area of 22.5 m²/g showed the formation of hydrogen, carbon monoxide, carbon dioxide and methane. Besides the production of hydrogen, carbon monoxide, carbon dioxide and methane, small amounts of ethene and ethane were detected on CeO₂ oxide with lower BET surface area (7 m²/g). Furthermore, for all H₂O/ethanol molar ratios studied, CeO₂ oxide with higher BET surface area showed the higher hydrogen and carbon monoxide production.

The performance of CuO, CuO/SiO₂, CuO/Al₂O₃ and CuO/CeO₂ oxides on steam reforming of ethanol was studied at 473 and 673 K, using a H₂O/ethanol molar ratio of 5 and 10 [T. Nishiguchi, T. Matsumoto, H. Kanai, K. Utani, Y. Matsumurab, W-J. Shenc, S. Imamura, Appl. Catal. A 279 (2005) 273]. Ethanol conversion was higher than 50% for CuO/Al₂O₃ and CuO/CeO₂ at temperatures higher than 520 K. Nevertheless, it is important to stress that the performance of these materials was evaluated using large amounts of catalysts (300-500 mg). Concerning selectivity to products, CuO, CuO/SiO₂ and CuO/Al₂O₃ exhibited low hydrogen formation. Moreover, it was observed a significant amount of acetaldehyde for CuO and CuO/SiO₂ catalysts and a large production of ethene on CuO/Al₂O₃ catalyst. In the case of CuO/Al₂O₃, the formation of by-products was attributed to acid sites of alumina. In order to minimize the by-products production, these sites were neutralized with a KOH solution. However, the sample treated with KOH presented high production of acetaldehyde. For CuO/CeO₂ catalyst, hydrogen and ketone were the main products formed. Nevertheless, the hydrogen production was slightly higher than that obtained for all catalysts. Acetaldehyde was also observed on CuO/CeO₂ catalyst. According to the authors, the acetaldehyde is produced on CuO and it was converted to ketone on CeO₂. The effect of the addition of MgO to the catalysts was also studied. The results showed that the presence of a basic oxide such as MgO, increased the formation of ketone and hydrogen. No comments were done about the stability of the catalysts and the production of carbon monoxide during the reaction.

According to the literature [J. Llorca, N. Horns, P. R. de Ia Piscina, J. Catal 227 (2004) 556; J. M. Guil, N. Horns, J. Llorca, P. R. de Ia Piscina, J. Phys. Chem. B 109 (2005) 10813; J. Llorca, P. R. de Ia Piscina, J. Sales, N. Horns, Chem. Commun. (2001) 641], among all oxides studied, ZnO presented the best performance on the steam reforming of ethanol at 598-673 K. At this temperature range, the reaction was performed using a mixture of water, ethanol and argon (ethanol/H₂O/Ar=1:3:20). The ethanol conversion was low (4.7-15.9%), in spite of the large amounts of catalysts used (300-500 mg). The main products obtained in dry base was hydrogen (45-51%), ethene (˜11-13%), acetaldehyde (˜20-40%) and ketone (˜3-9%). The ethanol conversion was high only at 673 K, using 100 mg of ZnO and a ethanol/(ethanol+H₂O) molar ratio of 5. Under these conditions, it was observed a high selectivity to hydrogen (61%). However, the formation of by-products such as ketone (9.2%), acetaldehyde (5.9%) and ethene (1.9%) was also detected. The formation of carbon monoxide was not observed. None of the works described above evaluated the stability of ZnO on steam reforming of ethanol.

Taking into account the results reported above, it was clear that a highly active, stable and selective catalysts is still unavailable and it must be developed in order to achieve an efficient process for hydrogen production through steam reforming and autothermal reforming of alcohols or a mixture of alcohols at low temperatures focused on minimizing the formation of by-products, such as ketone, acetaldehyde and ethene and deplete the CO production.

The catalysts of the present invention exhibited high activity and stability on steam reforming and autothermal reforming of alcohols or a mixture of alcohols at low temperatures, providing a process of hydrogen production with high selectivity to hydrogen, low formation of carbon monoxide (<150 ppm), small amounts of acetaldehyde and ethene and no production of ketone.

The main goal of this invention is to develop highly active and stable catalysts, which exhibit high selectivity to hydrogen, without CO formation, on steam reforming and autothermal reforming at low temperatures of alcohols, in particular ethanol, or a mixture of these alcohols, like, for example, bio-ethanol. The hydrogen produced is used as a fuel for a low temperature fuel cell like, for example, PEM fuel cell.

### BRIEF DESCRIPTION OF FIGURES

FIG. 1—Scheme of hydrogen production process for PEM fuel cells.

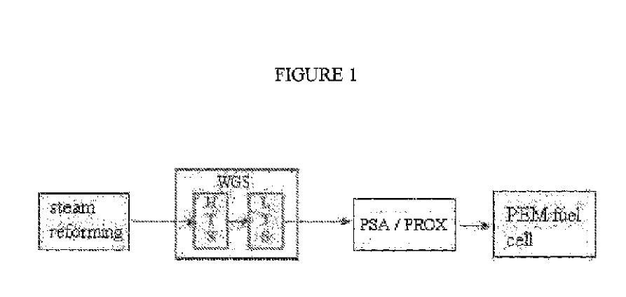

FIG. 2—FIG. 2 shows the ethanol conversion (Xₑₜₕₐₙₒₗ) as a function of time on stream on steam reforming of ethanol for CeO₂-A catalyst. Reaction conditions: T_{reaction}=773 K; H₂O/etanol molar ratio=2; m_{catalyst}=20 mg; W/Q=0.02 g.s/cm³.

FIG. 3—FIG. 3 presents the ethanol conversion (Xₑₜₕₐₙₒₗ) as a function of time on stream on steam reforming of ethanol for CeO₂—B catalyst. Reaction conditons: Tᵣₑₐₒₜᵢₒₙ=773 K; H2O/etanol molar ratio=2; m_{catalyst}=20 mg; W/Q=0.02 g.s/cm³.

FIG. 4—FIG. 4 shows the ethanol conversion (Xₑₜₕₐₙₒₗ) as a function of time on stream on steam reforming of ethanol for Ce_{0.75}Zr_{0.25}O₂ catalyst. Reaction conditions: T_{reaction}=773 K; H₂O/etanol molar ratio=2; m_{catalyst}=20 mg; W/Q=0.02 g.s/cm³.

### DETAILED DESCRIPTION OF THE INVENTION

This invention comprises the use of the cerium oxide based catalysts with or without 0.5-10 wt % of alkaline and alkaline earth promoters (Li, Na, K, Rb, Cs, Fr, Be, Mg, Ca, Sr, Ba, Ra) and mixed oxides containing ceria and zirconia and/or yttria an/or lanthanide elements (CeₓM₁₋ₓO₂, M=Zr, Y, La, Pr, Nd, Pm, Sm, Eu and 0.1<x<0.9) in the steam reforming and autothermal reforming at low temperatures of alcohols, in particular ethanol, or a mixture of these alcohols, like, for example, bio-ethanol. Low temperature was defined as 723-823 K.

The alcohols used in this invention containing one to five carbons (C₁₋₅ alcohols), such as, for example, methanol, ethanol, 1-propanol, iso-propanol, 1-butanol, 1-pentanol, or a mixture of alcohols, such as, for example, bio-ethanol. Preferred alcohol is methanol and particularly preferred is ethanol.

First of all, the preparation of cerium oxide used in the present invention is described.

The cerium oxide was obtained by three different methods.
(1) Method A: Calcination of (NH4)2Ce(NO3)6 in a muffle at temperatures between 673 an 1273 K, preferably between 700 and 1073 K and more preferably between 723 and 873 K for less than two hours, preferably for one hour.
(2) Method B: Preparation through a method proposed by Chuah et al [G. K. Chuah, S. Jaenicke, S. A. Cheong, K. S. Chan, Appl. Catal. A 145 (1996) 267]. At first, an aqueous solution of cerium (IV) ammonium nitrate and zirconium nitrate was prepared. Then, it was slowly added to a NH₄OH solution and the pH was maintained at 10-14, preferably at 11-12. After precipitation, the material was heated to 323-423 K, preferably between 363 and 373 K, and kept at this temperature for 24-120 hours, preferably for 72-100 hours. The precipitate was collected by a centrifuge. Finally, the material was washed until it achieves pH=7 and dried at 373-423 K, preferably between 383 and 403 K for 8-24 hours, preferably 10-14 hours. Then, the sample was calcined at 10 K/min, preferably less than 5 K/min and more preferably less than 2 K/min, up to 673-1273 K, preferably 700-1073 K, and more preferably 723-873 K, for 8-24 hours, preferably 10-14 hours.
(3) Method C: Precipitation of (NH₄)₂Ce(NO₃)₆ with urea. An aqueous solution of (NH₄)₂Ce(NO₃)₆ and urea was heated to 323-423 K, preferably 353-373 K, under stirring and kept at the final temperature for 20-72 hours, preferably 24-36 hours. The material was collected by a centrifuge and washed until it reaches a pH of 7.0. Then, the sample was dried at 363-423 K5 preferably 383-403 K5 for 8-24 hours, preferably 10-14 hours. Next, it was calcined at 10 K/min, preferably less than 5 K/min and more preferably less than 2 K/min, up to 673-1273 K5 preferably 700-1073 K5 and more preferably 723-873 K5 for 8-24 hours, preferably 10-14 hours.

The alkaline and alkaline earth promoters were added to the cerium oxide by the incipient wetness impregnation technique using an aqueous solution containing the precursor salts of alkaline and alkaline earth metals. Generally, a chloride or a nitrate of alkaline and alkaline earth metal was used as a precursor salt. The amount of alkaline and alkaline earth promoter added was 0.5 to 10 wt %, preferably 1.5 to 5 wt % and more preferably 1.0 to 2 wt %. After impregnation, the samples were dried at 363-423 K, preferably 373-393 K for 12-24 hours, preferably 16-20 hours. Then, they were calcined under air at 573-873 K, preferably 623-723 K, for more than 1 hour, preferably for 2 hours.

CeₓM₁₋ₓO₂ oxides were obtained by the precipitation method as described by Hori et al. [[CE. Hori, H. Permana, K. Y. Ng Simon, A. Brenner, K. More, K. M. Rahmoeller, D. Belton, Appl. Catal. B 16 (1998) 105]. An aqueous solution of ceria, zirconia and/or yttria and/or lanthanide elements precursors was prepared with the desired composition (0.1<x<0.9, preferably 0.25≦x≦0.75). Then, the ceria and zirconium an/or yttria and/or lanthanide hydroxides were co-precipitated by the addition of an excess of ammonium hydroxide. After filtration and washing with distilled water until the filtrate reaches a pH of 7.0, the samples were calcined in muffle at 673-1273 K, preferably 700-1173 K, for less than 2 hours, preferably for 1 hour.

Next, steam reforming and autothermal reforming of alcohols (C1 to C5), in particular ethanol, or a mixture of these alcohols, like, for example, bio-ethanol were performed, using the catalysts prepared by the methods described above. The reactions were carried out in a fixed bed reactor at atmospheric pressure for 3-36 hours, preferably 6-30 hours.

Prior to reaction, the catalysts were pretreated at different conditions, such as: (i) treatment under air at 673-1273 K, preferably 700-1173 K, for less than 2 hours, preferably for one hour; (ii) reduction under H2 at 473-873 K, preferably at 523-823 K, for less than 2 hours, preferably for one hour.

The reaction temperature is generally 723 to 823 K, preferably 773 K.

The feedstock contained a H2O/alcohol molar ratio between 0 and 15, preferably between 2 and 6.

The oxygen was introduced in the feed in order to have a O2/alcohol molar ratio of 0.1-5.0, preferably 0.5-1.0.

The residence time used (W/Q; W=mass of catalyst and Q=volumetric flow) was 0.01-0.08 g.s/cm³, preferably 0.015-0.03 g.s/cm³.

All catalysts exhibited good stability and high selectivity to hydrogen in the reaction conditions described above.

The present invention will be described by the following examples, which are provided for illustrative purposes only.

### Example 1

### Preparation of CeO2 Catalyst by Method A (CeO2-A)

The CeO₂-A catalyst was obtained through calcination of (NH₄)₂Ce(NO₃)₆ at 773 for 1 hour in muffle.

### Example 2

### Preparation of CeO2 catalyst by Method B (CeO2-B)

An aqueous solution with 10% wt of (NH₄)₂Ce(NO₃)₆) and an aqueous solution of NH₄OH (5M) were prepared. The solution of (NH₄)₂Ce(NO₃)₆ was slowly added to the solution of NH₄OH and the pH was adjusted in order to have an alkaline solution. After precipitation, the material was heated to 369 K and kept at the final temperature for 96 hours. Next, the sample was collected by a centrifuge, washed and dried at 393 K for 12 hours. Then, the material was calcined at 1 K/min up to 773 K and kept at this temperature for 12 h.

### Example 3

### Evaluation of the Stability of CeO₂-A Catalyst on Steam Reforming of Ethanol

The stability of CeO₂-A catalyst, which was prepared as described in the example 1, was evaluated on steam reforming of ethanol for 30 hours time on stream. The reaction was carried out in a fixed bed reactor at atmospheric pressure. Prior to reaction, the catalyst was reduced under H₂ at 573 K for 1 hour. The reaction was performed at 773 K5 using 20 mg of catalyst and W/Q=0.02 g.s/cm³. The reactants were fed to the reactor by bubbling N2 through two saturators (one of them containing ethanol and the other containing water) in order to obtain an ethanol:H2O:N₂ molar ratio=1:2:22.5.

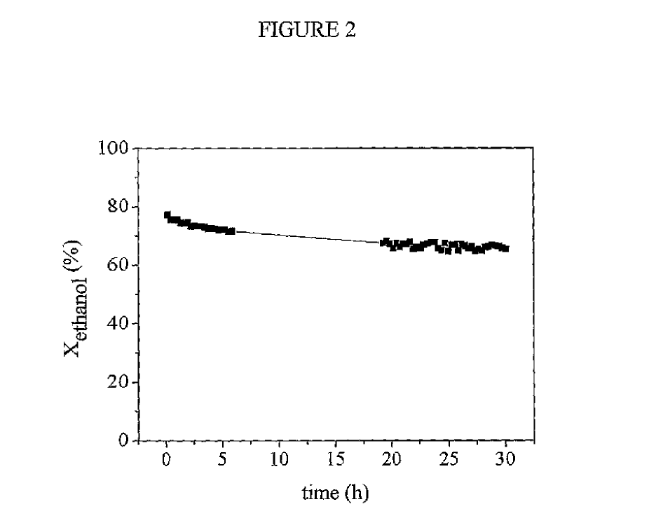

FIG. 2 shows the ethanol conversion (Xₑₜₕₐₙₒₗ) as a function of time on stream obtained on steam reforming of ethanol for CeO₂-A catalyst. The initial ethanol conversion was, approximately, 77%. It was also observed that, after an initial period of slight deactivation, the catalyst became practically stable (FIG. 2).

### Example 4

### Evaluation of the Performance of CeO2-B Catalyst on Steam Reforming of Ethanol

The performance of CeO₂—B catalyst, which was prepared as described in the example 2, was evaluated on steam reforming of ethanol. The reaction was performed at the same conditions described in example 3.

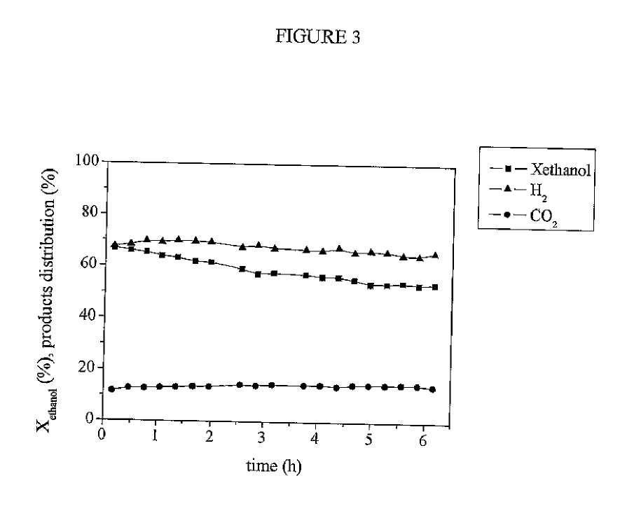

The ethanol conversion (Xₑₜₕₐₙₒₗ) and the products distribution as a function of time on stream for CeO₂—B catalyst are presented in FIG. 3.

The initial ethanol conversion was, approximately, 67%. Moreover, the CeO₂—B catalyst exhibited a slight deactivation in the beginning of the reaction, becoming stable after 4 hours time on stream. Hydrogen and carbon dioxide were the main products obtained. It was also observed the formation of small amounts of acetaldehyde and ethene. Furthermore, only traces of carbon monoxide were produced (˜150 ppm) and the formation of ketone was not detected.

### Example 5

### Preparation of Ce_{0.75}Zr_{0.25}O₂ Catalyst

For the preparation of CeₓM₁₋ₓO₂ catalyst with x=0.75 and M=Zr (Ce_{0.75}Zr_{0.25}O₂), an aqueous solution of cerium (IV) ammonium nitrate and zirconium nitrate was prepared with the Ce/Zr ratio of 3.0. Then, the ceria and zirconium hydroxides were co-precipitated by the addition of an excess of ammonium hydroxide. After filtration and washing with distilled water until the filtrate reaches a pH of 7.0, the sample were calcined at 1073 K for 1 hour in a muffle.

### Example 6

### Evaluation of the Stability of Ce_{0.75}Zro.25O2 catalyst on Steam Reforming of Ethanol

The stability of Ce_{0.75}Zr_{0.25}O₂ catalyst, which was prepared as described in example 5, was evaluated on steam reforming of ethanol for 30 hours time on stream. The reaction was carried out in a fixed bed reactor at atmospheric pressure. Prior to reaction, the catalyst was reduced at 773 K for 1 hour. The reaction was performed at 773 K5 using 20 mg of catalyst and W/Q=0.02 g.s/cm³. The reactants were fed to the reactor by bubbling N₂ through two saturators (one of them containing ethanol and the other containing water) in order to obtain a ethanol:H₂O:N₂ molar ratio=1:3:17.

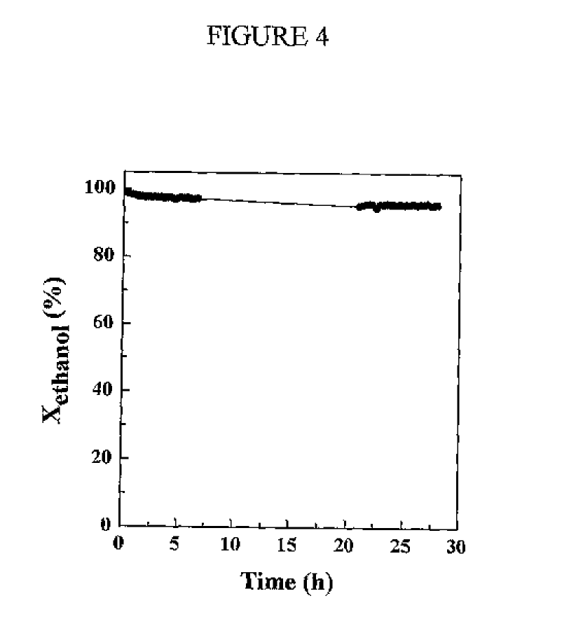

FIG. 4 shows the ethanol conversion (Xₑₜₕₐₙₒₗ) as a function of time on stream obtained for Ce_{0.75}Zr_{0.25}O₂ catalyst. The ethanol conversion was complete and the catalyst remained quite stable during 30 hours time on stream.

The examples reported above show that the catalysts of the present invention exhibit high activity and stability, high selectivity to hydrogen, low formation of carbon monoxide (<150 ppm), small amounts of acetaldehyde and ethene and no production of ketone.

1. Catalyst for steam reforming and autothermal reforming of alcohols at low temperature which comprises cerium oxide based materials.

2. Catalyst for steam reforming and autothermal reforming of alcohols at low temperature according to claim 1 which comprises cerium oxide based materials with or without 0.5-10 wt % of alkaline and alkaline earth promoters.

3. Catalyst for steam reforming and autothermal reforming of alcohols at low temperature according to claim 2 which comprises cerium oxide based materials with or without 1.5-5.0 wt % of alkaline and alkaline earth promoters.

4. Catalyst for steam reforming and autothermal reforming of alcohols at low temperature according to claim 3 which comprises cerium oxide based materials with or without 1.0-2.0 wt % of alkaline and alkaline earth promoters.

5. Catalyst for steam reforming and autothermal reforming of alcohols at low temperature which comprises CeₓM₁₋ₓO₂ mixed oxides wherein 0.1<x<0.9 and M=zirconia and/or yttria and/or elements of lanthanide group.

6. Catalyst for steam reforming and autothermal reforming of alcohols at low temperature according to claim 5 which comprises CeₓM₁₋ₓO₂ mixed oxides wherein 0.1<x<0.9 and M=Zr, Y, La, Pr, Nd, Pm, Sm, Eu.

7. Catalyst for steam reforming and autothermal reforming of alcohols at low temperature according to claim 5 which comprises CeₓM₁₋ₓO₂ mixed oxides wherein 0.25≦x≦0.75.

8. Use of the catalysts on steam reforming and autothermal reforming of alcohols at low temperature according to any one of claims 1 to 7, in the following reaction conditions: (i) reaction temperature from 723 to 823 K; (ii) H₂O/etanol molar ratio between 0 and 15; (iii) O₂/etanol molar ratio between 0.1 and 2.0 and (iv) residence time (W/Q) of 0.01-0.08 g.s/cm³.

9. Use of the catalysts on steam reforming and autothermal reforming of alcohols at low temperature according to any one of claims 1 to 7, in the following reaction conditions: (i) reaction temperature of 773 K; (ii) H₂O/etanol molar ratio between 2 and 6; (iii) O₂/etanol molar ratio between 0.5 and 1.0 and (iv) residence time (W/Q) of 0.015-0.03 g.s/cm³.

10. Steam reforming and autothermal reforming of alcohols at low temperature, which comprises the use of cerium oxide based catalysts according to claims 1, 5 and 8 and C₁₋₅ alcohols.

11. Steam reforming and autothermal reforming of alcohols at low temperature, which comprises the use of cerium oxide based catalysts according to claim 10, wherein the alcohol is methanol.

12. Steam reforming and autothermal reforming of alcohols at low temperature, which comprises the use of cerium oxide based catalysts according to claim 10, wherein the alcohol is the ethanol.
